(19) United States
(12) Patent Application Publication
(10) Pub. No.: US 2006/0223060 A1
(43) Pub. Date: Oct. 5, 2006

(54) Method for detecting analytes

(75) Inventors: Johannes Bonenberger, Kempten (DE); Sigrid Hagenmaier, Ulm (DE); Jitka Polackova, Neu-Ulm (DE); Jan Von Kampen, Vöhringen (DE)

Correspondence Address: FULBRIGHT & JAWORSKI L.L.P., 600 CONGRESS AVE., SUITE 2400, AUSTIN, TX, 78701, US

(73) Assignee: Cibitest Gmbh & Co. KG, Neu-Ulm (DE)

(21) Appl. No.: 10/529,700

(22) PCT Filed: Sep. 30, 2003
(86) PCT No.: PCT/DE03/03253

§ 371 Date: Apr. 7, 2006

(30) Foreign Application Priority Data
Sep. 30, 2002 (DE) 102 45 644.5

(51) Int. Cl. C12Q 1/68; G01N 33/543

(52) U.S. Cl. 435/6; 436/518

(57) ABSTRACT

The present invention relates to a method and devices for detecting analytes comprising the following stages: incubation of a sample with macromolecules, to each of which at least 2 molecules of the analyte to be detected in the sample are coupled; subsequent incubation of the sample with a solid carrier, to which capture molecules for the analyte to be detected are coupled; addition of a fluorescence dye to stain the macromolecules and detection of analytes present in the sample by excitation of the fluorescence dye.

The present invention relates to a method and devices for detecting analytes comprising the following stages: incubation of a sample with macromolecules, to each of which at least 2 molecules of the analyte to be detected in the sample are coupled; subsequent incubation of the sample with a solid carrier, to which capture molecules for the analyte to be detected are coupled; addition of a fluorescence dye to stain the macromolecules and detection of analytes present in the sample by excitation of the fluorescence dye.

Numerous methods have already been established for detecting low-molecular-weight analytes. These methods are used in special laboratories, for example, in the quest for active ingredients for the pharmaceutical industry in the context of so-called high-throughput-screening (HTS) or very-high-throughput-screening (VHTS), and also for in-situ testing systems, for example, rapid drugs tests, environmental tests or food tests. In this context, some hundred (HTS) to several thousand (VHTS) “capture molecules”, also referred to as target molecules, generally DNA or proteins, are immobilised on slides in so-called microarrays, a distinction being currently made primarily between DNA arrays and protein arrays dependent upon the type of immobilised molecules. The interaction of the immobilised biomolecules can be investigated with the previously marked contents of an investigation matrix, for example, the lysate of a given tissue type.

In the case of HTS or VHTS, the detection methods are often based on fluorescence marking. These methods have been known in chemical, biochemical and biological analysis for a considerable time, for example, through the use of FIAs (Fluorescent Immuno Assay), which are implemented on microtitre plates or on biochips in slide format. The fluorescence dyes used in this context are generally very short-lived, require intensive excitation with a defined wavelength and generally emit at a nearby wavelength, so that an optical filter has to be placed between excitation and emission, and the emitted light has to be registered by an optical evaluation unit (fluorescence reader etc.). In view of the minimal quantities of the analytes used and their spatial density, the evaluation of the microarrays requires high-precision optical instruments, for example, fluorescence microscopes and computer-assisted image-evaluation software.

The currently available fluorescence-marking systems are not currently used for in-situ test systems, for example, rapid drugs tests and environmental tests because of the cost-intensive and sensitive detection technology. Test systems, which can be implemented and evaluated in-situ are characterised by their robustness when being used by un-trained personnel, under harsh environmental conditions, and also by the possibility for direct visual evaluation by the human eye. The marking methods currently available for such test systems are the coupling of biomolecules to gold particles, coloured polystyrene or latex beads, liposomes or enzymes. All of these marking methods require a white or the lightest-possible carrier material, on which the reaction partners are immobilised, in order to provide the maximum possible contrast after the reaction with a marked reactant. Accordingly, the markings currently available for rapid analysis cannot be realised for arrays on clear and/or transparent carrier materials. A further disadvantage of these visually-evaluated marking methods is the difficulty of evaluation in the dark and/or in artificial light. These are essential requirements for many applications, such as late-night drugs testing of motorists or the evaluation of hazardous-substance tests in the food industry, for example, in the environmental conditions of an animal stall.

The present invention is therefore based on the object of providing a sensitive and rapid method for the detection of chemical substances, which can be implemented simply in-situ and under unfavourable conditions.

This object is achieved by the subject matter defined in the claims.

The following drawings explain the invention.

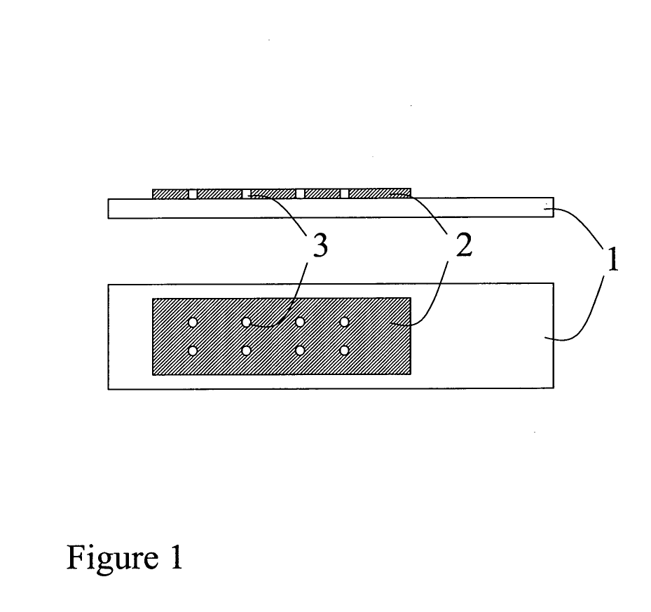

FIG. 1 shows a test slide in cross-section and viewed from above. The actual test slide (1) is provided with a coating (2), in which the capture molecules (3) are bound.

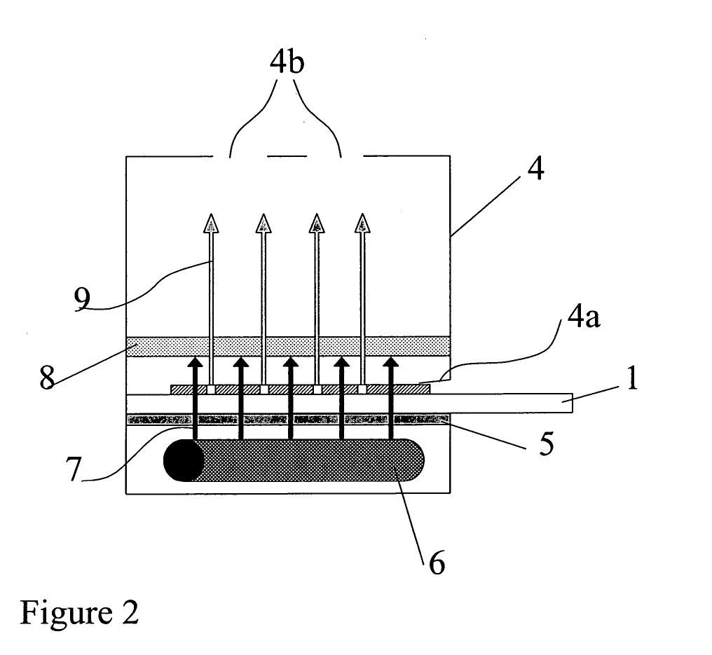

FIG. 2 shows an apparatus, with which the test slides can be evaluated after the test procedure. The test slide (1) is pushed along a guide (4a) into the housing (4) of the apparatus. In the apparatus, a light source (6) and a filter plate (5), which only allows the penetration of light within the range of the excitation maximum (7) of the fluorescence dye used for the detection, are disposed below the test carrier. A second filter plate (8), which blocks the light for the excitation (7) and allows the penetration of the light (9) emitted by the fluorescence dye, is disposed above the test slide. The positions, at which a fluorescence emission occurs on the test slide, can be observed through the apertures (4b) in the housing either visually or using appropriate optical instruments in dependence upon the dye used.

Within the present document, the term “analyte” refers to the substance to be investigated using the present method.

The term “capture molecule” or “target molecule” as used in the present document refers to the molecule applied according to the present method to the coating of the solid carrier, to which a free or coupled analyte can bind.

Methods and devices are described below, which allow the detection of analytes, preferably low-molecular-weight analytes, using fluorescence dyes, in order to achieve the object of the invention.

The present invention relates to a method for detecting analytes comprising the following stages:

a) Incubation of a sample with macromolecules, to each of which at least 2 molecules of the analyte to be detected in the sample are coupled;

b) Subsequent incubation of the sample with a solid carrier, to which capture molecules for the analyte to be detected are coupled;

c) Addition of a fluorescence dye to stain the macromolecules;

d) Detection of analytes present in the sample by excitation of the fluorescence dye.

With the method according to the invention, the analytes present in the sample, which are to be detected, compete for binding to the capture molecules with the molecules identical to the analytes, which are bound to the macromolecules. If the analytes to be detected are present in the sample, the analytes bound to the macromolecules are prevented from binding to the capture molecules. An excitation of the fluorescence dyes therefore fails to produce a positive signal. By contrast, if none of the analytes to be detected are present in the sample, the analytes bound to the macromolecules bind to the capture molecules, and the excitation of the fluorescence dye leads to a positive signal.

After stage c), a further stage c′): Removal of the non-bound fluorescence dyes from the solid carrier, can optionally be inserted.

Furthermore, the macromolecules in stage a) can be marked with a fluorescence dye, so that stage c) and stage c′) can be omitted. In this case, after stage a), the further stage a′): Removal of non-bound macromolecules, can optionally be inserted.

The macromolecules can be the same or different (identical or non-identical). Furthermore, identical or non-identical analytes can be coupled to the macromolecules. Accordingly, the present invention also relates to a method, in which either identical macromolecules are used, to which identical or non-identical analytes are coupled, or in which non-identical macromolecules are used, to which identical or non-identical analytes are coupled. The number of non-identical analytes is not limited; in fact, the number of analytes depends upon the size of the macromolecules and on the coupling positions, to which the analytes are attached.

The analytes to be detected with the method according to the invention can be proteins, e.g. antibodies, hormones, e.g. growth hormones such as somatotropin, polypeptides, especially with a molecular weight of less than approximately 5000 Dalton, antibiotics, e.g. β-lactams, cephalosporines, sulfonamides, tetracyclines and other narcotics, especially illegal narcotics, such as tetrahydrocannabinol, cocaine, morphine, amphetamines, metamphetamines and others, vitamins, especially vitamin groups A-U, e.g. vitamin B12, biotin and other anabolic steroids such as testosterone, nonsteroidal androgens such as zeranol and others, pesticides, e.g. atrazine, PCB and others, doping agents, e.g. narcotics such as amphetamine, cerebral stimulants of the amine group such as ephedrine, psychoactive drugs such as 1,4-benzodiazepine, and others, or potential biological weapons, e.g. anthrax, botulinum toxin and others. By preference, the analytes to be detected are low-molecular-weight analytes, especially with a molecular weight less than approximately 5000 Dalton, especially less than approximately 1000 Dalton.

For the method according to the invention, the analytes to be detected are coupled to a macromolecule. In order to increase the sensitivity of the assay, at least two analytes are coupled to each macromolecule; however, there is no upper limit to the number of analytes per macromolecule. On the contrary, the number of analytes depends upon the size of the macromolecule and on the coupling positions, to which the analytes are attached. In the case of coupling to nucleic acids, e.g. oligonucleotides, the degree of coupling can therefore be varied over the sequence of bases suitable for coupling. If polypeptides are used as the macromolecules, the frequency of occurrence of amino acids suitable for the coupling in the primary sequence also influences the degree of coupling. In the case of a sequence of always the same nucleotide or the same amino acid, to which the coupling can be made, the molar relationship of macromolecule to analyte determines the degree of coupling. The molar relationship in this context can vary between 1:2 and approximately 1:10. Molar relationships of >1:10 may also be adequate and, in coupling to proteins, depend upon the tertiary structure.

The macromolecules suitable for the method according to the invention can be synthetic materials, for example, melamine resins, polyamino acids, e.g. polylysine or polyglutamate, peptides, polypeptides, proteins, e.g. antibodies, peptide nucleic acids or nucleic acids, e.g. DNA or RNA. The nucleic acids can be used as a single strand (ss) or double strand (ds). If DNA is used, the length and the secondary structure of the DNA can have a decisive influence on the sensitivity of detection. Double-strand and high-molecular-weight DNA is generally easier to stain than short, single DNA strands, however, the analytes can be coupled more simply via the exposed primary amino groups of adenine, guanine and cytosine in single-strand DNA. Accordingly, single-strand DNA is preferable as the macromolecule for the method according to the invention. Oligonucleotides are particularly preferred, wherein oligonucleotides with a length within the range from approximately 40-80 bases are preferred for reasons of cost. Oligonucleotides with a length of less than approximately 40 bases are also suitable, but do not produce an optimum signal amplification. Oligonucleotides with a length greater than approximately 80 bases are also suitable, but are not preferred for reasons of cost. The oligonucleotides coupled to the analytes can optionally be hybridised with a complementary oligonucleotide, in order to obtain the signal strength of a double-strand DNA. To avoid self-hybridisation of single-strand oligonucleotides, a sequence of non-complementary bases is preferred.

The analytes are preferably coupled to the macromolecules via primary amino groups, especially via the amino groups of the nucleic acid bases. The coupling to peptides or proteins can also be via sulfhydryl groups and carboxy groups, and, in the case of glycosylated proteins can be formed via the sugar residue. In this context, the couplings are particularly formed via glutardialdehyde, succinimide ester, sulfo-succinimides, arylazides, phenyl azides, maleimides after previous conversion of the amino group into a sulfhydryl group, hydrazides, pyridil thio compounds after conversion of the amino group into a sulfhydryl group, imidoesters, carbodiimides, compounds containing vinyl-sulfone-reactive groups after conversion of the amino group into a sulfhydryl group, compounds containing boric acid for coupling to cis-diols in ribonucleotides instead of the amino group in the bases, conversion of the primary amino groups into carboxyl groups and subsequent coupling, hydroxy phenylazides, isocyanates, azidomethyl coumarins, nitrophenyl azides, bromoacetyl compounds, perfluoroazide compounds, iodoacetyl compounds, psoralenes, iodoacetate compounds, biotin-containing compounds, hydroxymethyl phosphines, aldehyde and hydrazide-activated dextrans. In this context, spacers or polylinkers of different lengths can be introduced between the molecules to be coupled, which can also influence the solubility of the coupled molecules in organic solvents.

The degree of coupling of an analyte can be varied through the selection of the nucleic acid sequence. For analytes with a molecular weight of approximately 300 Dalton, it has been shown that one coupling option to every fifth base in the molecule is adequate. However, more or less couplings can also be present. The length of the oligonucleotide and the degree of conjugation have an influence on the sensitivity of the detection system and can be determined empirically in each case.

Moreover, the present invention relates to a method, wherein the macromolecules added to the sample are coupled to different analytes. In this context, identical macromolecules can be coupled to non-identical analytes, and non-identical macromolecules can also be coupled respectively to non-identical analytes. This method can be used, e.g. for the detection of several analytes in a multiple test (array). By preference, a multiple test consists of identical macromolecules, to which non-identical analytes are coupled. In this context, the analytes must be used for coupling to the macromolecule in relationship to the desired sensitivity of detection. If oligonucleotides are used as the macromolecules, the oligonucleotides can preferably be manufactured with non-identical analytes, by using nucleotides in the synthesis of the oligonucleotides, to which non-identical analytes have been coupled before synthesis.

The fluorescence dyes required for the detection can either be added to the sample incubated with the macromolecules in solution or can already been coupled to the macromolecules. Fluorescence dyes are already known from molecular biology, with which DNA can be detected in a highly-sensitive manner within the range of a few picograms even with the naked eye after excitation with the appropriate wavelength, if this is focused to a narrowly limited range by appropriate means (e.g. by gel electrophoresis or fractionation in the CsCl gradient).

Currently available dyes for staining DNA are ethidium bromide, which is a member of the group of phenanthrenes, and which emits in the red range after excitation in the UV range, or the SYBR dyes (SybrGreen or SybrGold) manufactured by Molecular Probes, which are members of the group of cyanines and can be excited with UV at a maximum of 485 nm, and which emit at 530 nm. Both of these dyes are suitable for marking single-strand DNA or RNA, ethidium bromide being less suitable for in-situ test methods because of its carcinogenicity and because the UV excitation used is hazardous for unprotected eyes. Intercalating dyes, such as the phenanthrenes (e.g. propidium iodide, hexidium iodide, dihydroethidium, ethidium homodimers, ethidium monoazide) and acridines (e.g. acridine orange) and the indoles and imidazoles binding in the shallow groove of the DNA (e.g. bisbenzimide-dyes manufactured by Hoechst) and other dyes such as 7-aminoactinomycin D, actinomycin D and hydroxystilbamidine, are suitable for marking double-strand DNA or RNA.

According to one preferred embodiment of the method according to the invention, double-strand DNA, which binds more fluorescence dye when intercalating dyes are used, is produced by hybridisation of oligonucleotides with complementary DNA sequences, thereby increasing the sensitivity of the method according to the invention.

Linker-coupled fluorophores, such as n-hydroxy succinimide esters of all currently available fluorescence dyes, e.g. manufactured by Molecular Probes, are suitable for staining polyamino acids. These dyes show a great variability of the fluorophores, e.g. from the blue-fluorescing dyes Cascade Blue, Marina Blue and Alexa Fluor 350 to the green-fluorescing dyes Alexa Fluor 488, Alexa Fluor 532, Alexa Fluor 546, Alexa Fluor 568, the red-fluorescing dyes Alexa Fluor 594, Texas Red and Texas Red-X and the dark-red-fluorescing dyes Alexa Fluor 633, Alexa Fluor 647, Alexa Fluor 660, Alexa Fluor 680 and LaserPro IR 790 dyes and the phycobilins.

The above list of dyes is not exclusive and is mentioned here only by way of example. It is evident to a person skilled in the art, that other dyes, which stain the macromolecules used according to the method, can also be used.

The solid carriers used for the method according to the invention can either be light-permeable, solid carriers, e.g. slides made from glass and/or polymer synthetic materials, such as polystyrene or others, or light-impermeable carriers such as synthetic-material strips, coated with membranes, e.g. nitrocellulose, nylon and others. In one preferred embodiment, the solid carrier can also be a transparent column, which is filled with antibody-binding materials, or a rod-shaped synthetic-material carrier, which is suitable for binding antibodies as a result of chemical modifications, e.g. amination and others, or physical modifications, e.g. fusion of Eupergit and others.

The capture molecules coupled on a solid carrier can be any kind of molecule, which can bind specifically to the analytes to be detected. Preferred capture molecules are antibodies, receptors, molecular-imprinted polymers (molecular imprints) and their counterparts, such as antigens, ligands, functional monomers and/or cross-linked monomers (printing molecules).

The capture molecules can be coupled on commercially available, specially pre-coated slides, e.g. epoxy or aldehyde slides (Quantifoil Micro Tools GmbH, Jena), Euray (Exiqqon, Vedbaek, Denmark) and others, the procedure being carried out according to the manufacturers' instructions. Pre-coated slides are also suitable, e.g. coated with oxirane-containing dispersion (Preparation 2879, Röhm Pharma, Darmstadt). For this purpose, the oxirane-containing dispersion can be painted onto a slide and dried for some hours at room temperature. In order to obtain a signal, which is readily visible to the eye, the specific antibodies are spotted onto the prepared slides in a volume of approximately 0.01 to approximately 10 μl. The volume varies depending upon the coating of the slide, the number of antibodies to be spotted and the desired magnitude of the signal. By preference, the volumes are between approximately 0.1 and approximately 0.5 μl per spot, with a titre greater than 1 of the antibody solution. The spotted antibodies are preferably dried for several hours at 37° C., following this, the free, non-spotted surfaces of the slide coating can be blocked with casein or other suitable proteins such as BSA (respectively 1-10%) in physiological buffers, e.g. PBS or animal sera (pig, hen etc. 10% in PBS) according to general specifications for immuno-detection, e.g. Application Guide Pall Gelman Laboratory: Transfer and Immobilisation Procedures for Pall Gelman Laboratory Membranes. Non-bound protein is washed away, and the slides are now activated and can be used for detecting the antibody-specific analytes. In one specific embodiment, 10-100 ng of a freeze-dried, analyte-coupled oligonucleotide are placed in a test vessel. This oligonucleotide is dissolved with the sample, e.g. with one millilitre of a sample containing the free analyte, and the activated slide is incubated in the test liquid for a defined period, dependent on the affinity of the antibody, between 0.5 and 120 minutes with an ambient temperature of 5 to 35° C.

The competition between the free analytes and the analytes bound to the macromolecule for the binding positions of the antibody takes place during the incubation period, wherein the sensitivity of the test for the free analytes can be adjusted via the quantity of macromolecule-bound analytes. Depending on the choice of fluorescence dye, the macromolecule, e.g. the oligonucleotide, may already be pre-stained, or staining may be required for a defined period after incubation and rinsing of the slide, e.g. in a separate test vessel with a defined concentration of dye. Surplus dye can be removed after staining in order to reduce the background. This can be achieved either mechanically by scraping, e.g. against a rubber lip, or by means of a defined volume of a rinsing fluid. The composition of the rinsing fluid depends upon the stability of the bound capture molecules, wherein aqueous solutions with buffer properties appropriate for the capture molecules and for the other reaction partners used are preferred. The detection of the macromolecule-bound analytes takes place following this by excitation of the fluorescence dyes through the slide from below. In one preferred embodiment, the oligonucleotide used as the macromolecule is stained with SYBR-Gold (Molecular Probes), excited with a blue-light transilluminator (450 nm) and the fluorescence radiation is made visible to the human eye through an orange filter (500-510 nm).

In the case of a detection system for in-situ use, a battery-operated, blue-light transilluminator is preferably used. This is fitted with an orange filter with a shade mounted above this. In its dimensions, the screen area of the transilluminator corresponds to the area of a slide used for the test. The filter is fitted above the screen surface in such a manner that the slide is pushed between the screen surface and the filter and can be fixed by spring clips or similar holding devices. In order to perceive the full intensity of the fluorescence radiation with the eye, the filter is connected at the light-emitting side to a light-impermeable mask adapted to the shape of the face, which fits against the eye region of the operator and excludes daylight or light interference from the surroundings.

The method can be used for testing foodstuffs and animal fodder, e.g. for the detection of hazardous substances, e.g. antibiotics, pesticides, hormones etc. Applications such as these have so far relied exclusively on laboratory methods, which are so time-consuming that the foodstuffs or animal fodder have already been consumed before the results from the investigation are available. Moreover, the markings already used for rapid immunological testing are of limited suitability for in-situ evaluation, for example, in artificial light in the supermarket or when carrying out tests in poorly illuminated animal stables. This problem is resolved by the present invention.

Further exemplary uses, to which the method according to the invention brings considerable advantages, are to be found within the field of diagnostics. The method provides considerable advantages in veterinary diagnostics under animal-keeping conditions and also in emergency human diagnostics, where rapid tests have to be carried out around the clock in an ambulance.

Moreover, the method according to the invention is particularly suitable in the context of traffic checks for registering motorists under the influence of drugs. The problem of drug consumption is often associated with late-night visits to discotheques. Accordingly, the tests are carried out predominantly under unfavourable lighting conditions. The evaluation of rapid tests for illegal drugs and barbiturates is therefore made considerably easier.

The following examples explain the invention but should not be regarded as restricting its scope.

(1) Manufacture of an Oligonucleotide

In order to achieve a targeted coupling, a sequence consisting of thymidine and guanosine was synthesised, since no analytes can be coupled to thymine because of the absence of a primary amino group. The coupling was therefore made by a primary amino group of the guanine. Cytosine would have been equally suitable for the coupling. The oligonucleotide with the sequence (TTTTG)₁₆ with a salt-free, standard level of purity was ordered from Qiagen Operon and supplied in freeze-dried form. A stock solution of 10 mg/ml in PBS (Phosphate Buffered Saline, 137 mM NaCl, 2.7 mM KCl, 8 mM Na₂HPO₄.2H2O; 1.5 mM KH₂PO₄) was prepared from the freeze-dried products, and stored in aliquots at −20° C.

(2) Coupling of Analytes

(a) Ampicillin to an Oligonucleotide,

for the activation 1.5 mg ampicillin (5 mg/ml in 100 mM c) was added drop-wise under constant stirring to 25 mg/ml m-maleimidobenzoyl-N-hydroxysulfosuccinimide ester (in H₂O) up to a final concentration of 5 mg/ml and then stirred for 30 minutes at room temperature. The surplus maleimide groups were inactivated by subsequent incubation for 1 hour after the addition of 1 M dithiothreitol up to a final concentration of 35 mM. For the coupling, 3.9 mg of the oligonucleotide (2 mg/ml in PBS) was added to the mixture, mixed and incubated for 3 hours at room temperature. In a subsequent dialysis against PBS carried out overnight with four-fold change of buffer 4° C., non-coupled ampicillin and m-maleimidobenzoyl-N-hydroxysulfosuccinimide ester were removed from the reaction mixture.

(b) Ampicillin and AlexaFluor 488 to Polylysine

Once again, for the activation, 2 mg ampicillin (5 mg/ml in 100 mM sodium hydrogen phosphate in H₂O, pH 7.2) were added drop-wise while stirring to 2 mg m-maleimidobenzoyl-N-hydroxysulfosuccinimide ester (25 mg/ml in H₂O) and then stirred for 30 minutes at room temperature. The surplus maleimide groups were inactivated by incubation for 1 hour with 1 M dithiothreitol (final concentration 35 mM). At the same time, 1 mg polylysine (3-15 kDa) was dissolved in 0.1 M sodium hydrogen carbonate (2 mg/ml); 4.4 μl AlexaFluor 488 were diluted to 100 μl with bi-distilled water, added in aliquots of 10 μl -20 μl while stirring and then stirred for 1 hour in darkness at room temperature. Free dye was separated with a 3K centrifugal concentrator (Pall) by centrifuging for 20 minutes at room temperature and washing once with 500 μl PBS. The concentrate was dissolved in 500 μl PBS. Following this, activated ampicillin was added and stirred for 3 hours in darkness at room temperature. Non-coupled ampicillin and m-maleimidobenzoyl-N-hydroxysulfosuccinimide ester was removed by dialysis against PBS carried out overnight at 4° C. and with four-fold change of buffer.

(3) Coating the Slide

Slides, e.g. “SuperFrost” manufactured by Menzel, were coated with 5 μl of an oxirane-containing dispersion (preparation 2879, Röhm Pharma, Darmstadt) by stroking evenly with a glass or plastic rod and then dried at room temperature for at least 2 hours. The antibodies were applied manually with a pipette in dilutions (10°, 10⁻¹, 10⁻², 10⁻³), applying volumes from 0.1 to 1.0 μl in such a manner that the tip of the pipette did not touch the slide. PBS proved very suitable as a diluent and application solution. The antibodies were fixed to the slide over 2-3 hours at 37° C., or overnight at room temperature. Following this, the coated slides were blocked at room temperature for 30 minutes in milk buffer (10 mM Tris-HCl, pH 7.4, 150 mM NaCl, 0.3% casein after Hammarsten, 0.05% Tween 20, 0.005% NaN₃ (optional)) or 100 mM Tris-HCl, pH 9 with 50 mM ethanolamine and 0.1% SDS or pig serum (1:10 diluted with PBS).

(4) Implementation of the Test

(a) Oligonucleotides Coupled with Ampicillin

The slides were incubated for 20 min. at room temperature lying in a flat dish with 20 ml 0.5% bovine serum albumin, 0.05% Tween 20 in PBS, with different concentrations of ampicillin and of the ampicillin-coupled oligonucleotide, and then washed briefly 3× with 11.5 mM NaCl, 0.23 mM KCl, 0.66 mM Na₂HPO₄×2H₂O, 0.13 mM KH₂PO₄, 0.04% Tween 20 in H₂O. Following this, the slides were stained for 1 minute with SYBRGold (Molecular Probes) in a dilution of 1:30,000 in TAE-buffer (40 mM tris-HCl, pH 7.5, 20 mM sodium-acetate, 1 mM EDTA). Immediately after staining, the slides were again washed as before, immediately placed into a blue-light transilluminator and evaluated. A negative result, that is, the absence of the analyte is indicated by a fluorescence signal. The intensity of the signal declines as the concentration of the analyte increases. The ampicillin conjugate was completely displaced at a concentration of 100 ng/ml ampicillin.

(b) Polylysine Coupled with Ampicillin and Fluorophore

Coated slides were incubated as described under (a) with various concentrations of ampicillin and the ampicillin-AlexaFluor488-coupled polylysine for 20 minutes under protection from light and rinsed as defined under (a). The slides were then placed without further treatment onto a blue-light transilluminator and evaluated. The absence of the analyte was indicated by a fluorescence signal. Increasing concentrations of the free ampicillin displaced the ampicillin conjugate until at 1 mg/ml ampicillin, no further signal could be observed.

(5) Results of Comparative Experiments

| | | | | | Test |
|---|---|---|---|---|---|
| | | Macromolecule | | | sensitivity |
| Detection of | Capture | (coupled | Fluorescence | | achieved so |
| free analyte | molecule | analyte) | dye | Test matrix | far |
| Ampicillin | Anti- | ssDNA- | SybrGold | buffer | <1 μg/ml |
| | ampicillin- | ampicillin | | | |
| | antibody | | | | |
| Ampicillin | Anti- | ssDNA- | SybrGold | milk | <10 μg/ml |
| | ampicillin- | ampicillin | | | |
| | antibody | | | | |
| Ampicillin | Anti- | ssDNA- | Ethidium- | buffer | <1 μg/ml |
| | ampicillin- | ampicillin | bromide | | |
| | antibody | | | | |
| Ampicillin | Anti- | dsDNA- | Ethidium- | buffer | <0.1 μg/ml |
| | ampicillin- | ampicillin | bromide | | |
| | antibody | | | | |
| Ampicillin | Anti- | Polylysine- | AlexaFluor- | buffer | <1 μg/ml |
| | ampicillin- | ampicillin | 488 | | |
| | antibody | | | | |
| Biotin | Anti-biotin- | ssDNA-biotin | SybrGold | milk | <1 μg/ml |
| | antibody | | | | |
| THC | Anti-THC- | Polylysine- | AlexaFluor- | buffer | <1 μg/ml |
| | antibody | THC | 488 | | |
| Skin-fungus- | Fungal | Anti-Ig- | AlexaFluor- | rabbit- | not measured |
| epitopes | antigen + anti- | antibody | 488 | serum | |
| | fungal | | | | |
| | antibody | | | | |

1. A method for detecting analytes comprising the following steps of:
a) incubating a sample with macromolecules, to each of which at least 2 molecules of the analyte to be detected in the sample are coupled;
b) further incubating the sample with a solid carrier, to which capture molecules for the analyte to be detected are coupled;
c) adding a fluorescence dye to stain the macromolecules; and
d) detecting the analytes present in the sample by excitation of the fluorescence dye.

2. The method according to claim 1 comprising, after step c), a further step c′): removing the non-bound fluorescence dye from the solid carrier.

3. A method for detecting analytes comprising the steps:
a) incubating a sample with fluorescence-dye-marked macromolecules, to each of which at least 2 molecules of the analyte to be detected in the sample are coupled;
b) further incubating the sample with a solid carrier, to which capture molecules for the analyte to be detected are coupled; and
c) detecting analytes present in the sample by excitation of the fluorescence dye.

4. The method according to claim 3 comprising, after step a), a further step a′): removing the non-bound macromolecules.

5. The method according to claim 1, wherein the macromolecules are nucleic acids, peptide nucleic acids, polyamino acids.

6. The method according to claim 1, wherein the macromolecules are single-strand oligonucleotides of a length within the range from 40 to 80 nucleotides.

7. The method according to claim 1, wherein the macromolecules are identical or non-identical.

8. The method according to claim 1, wherein the analytes have a molecular weight of less than 5000 Dalton.

9. The method according to claim 1, wherein the fluorescence dye is selected from the group of phenanthrenes, acridines, SYBR dyes or fluorophores.

10. The method according to claim 1, wherein the solid carrier is permeable to light and the detection method is implemented by means of a transmitted-light method.

11. A device comprising a light source fitted on one side of a solid carrier inserted into the device, that a filter disposed respectively between the light source and the solid carrier on the other side of the solid carrier, wherein the device is designed in such a manner that light passing through the solid carrier passes through an aperture into the human eye or into an optical instrument.

12. The method according to claim 3, wherein the macromolecules are nucleic acids, peptide nucleic acids, polyamino acids.

13. The method according to claim 3, wherein the macromolecules are single-strand oligonucleotides of a length within the range from 40 to 80 nucleotides.

14. The method according to claim 3, wherein the macromolecules are identical or non-identical.

15. The method according to claim 3, wherein the analytes have a molecular weight of less than 5000 Dalton.

16. The method according to claim 3, wherein the fluorescence dye is selected from the group of phenanthrenes, acridines, SYBR dyes or fluorophores.

17. The method according to claim 3, wherein the solid carrier is permeable to light and the detection method is implemented by means of a transmitted-light method.
